(19) United States
(12) Patent Application Publication
(10) Pub. No.: US 2024/0286139 A1
(43) Pub. Date: Aug. 29, 2024

(54) APPARATUS AND METHOD FOR TRANSFERRING A FLUID SAMPLE FROM A FLUID SAMPLE COLLECTION APPARATUS TO A LIQUID SAMPLE ANALYZER

(71) Applicant: Siemens Healthcare Diagnostics Inc., Tarrytown, NY (US)

(72) Inventors: Janine Cox, Stoughton, MA (US); Michael Chen, Sharon, MA (US); Justin Davis, Raleigh, NC (US); Justin DeSilva, Mendon, MA (US); Mark Vaughan, Foxborough, MA (US); Joshua Joseph, Apex, NC (US)

(73) Assignee: Siemens Healthcare Diagnostics Inc., Tarrytown, NY (US)

(21) Appl. No.: 18/681,802

(22) PCT Filed: Aug. 5, 2022
(86) PCT No.: PCT/US2022/074582

§ 371 Date: Feb. 6, 2024

Related U.S. Application Data
(60) Provisional application No. 63/232,365, filed on Aug. 12, 2021.
(60) Provisional application No. 63/244,987, filed on Sep. 16, 2021.
(60) Provisional application No. 63/366,558, filed on Jun. 17, 2022.
(60) Provisional application No. 63/367,239, filed on Jun. 29, 2022.
(60) Provisional application No. 63/369,522, filed on Jul. 27, 2022.

(51) Int. Cl. B01L 3/00

(52) U.S. Cl. CPC B01L 3/5635; B01L 2200/026; B01L 2300/0681; B01L 2300/069; B01L 2300/0832

(57) ABSTRACT

An apparatus and method for transferring a fluid sample from a fluid sample collection apparatus to a liquid sample analyzer. The apparatus includes a barrel having a first end, a second end, a sidewall extending between the first end and the second end, an inner surface defining an internal chamber, and an external surface defining at least a portion of a chromatographic assay chamber in fluid communication with the internal chamber. The first end has an inlet opening with a clot catcher extending across the inlet opening upstream of the passage and the second end has an outlet opening. A chromatographic assay assembly is housed in the chromatographic assay chamber for detecting presence of free hemoglobin in the fluid sample.

### CROSS-REFERENCE TO RELATED APPLICATIONS

This application claims benefit under 35 USC § 119(e) of U.S. Provisional Application No. 63/369,522, filed Jul. 27, 2022; U.S. Provisional Application No. 63/367,239, filed Jun. 29, 2022; U.S. Provisional Application No. 63/366,558, filed Jun. 17, 2022; U.S. Provisional Application No. 63/244,987, filed Sep. 16, 2021; and U.S. Provisional Application No. 63/232,365, filed Aug. 12, 2021. The entire contents of the above-referenced patent application(s) are hereby expressly incorporated herein by reference.

### BACKGROUND

Blood sampling is a common health care procedure typically used in hospital and laboratory settings to determine the physiological and biochemical condition of a patient. Blood sampling is essential to the diagnosis and treatment of patients suspected of a wide variety of disorders. Blood samples are analyzed by fluid testing devices, such as blood analyzers, to detect clinically significant variations in blood components, e.g., plasma, red blood cells, white blood cells, and platelets, or other characteristics, such as blood gas conditions. Analysis of a blood sample for blood gas conditions provides information regarding the amount of oxygen and carbon dioxide in the blood, and may also be used to determine the pH of a blood sample. Imbalances in the oxygen, carbon dioxide, or PH levels of a blood sample may indicate particular pathological conditions or stage of disease progression.

Blood samples are typically collected using blood collection syringes having hypodermic needles, or vacuum tubes coupled to a needle assembly. However, blood collection syringes are prone to have bubbles of air or other gases within the syringe. The bubbles in the barrel and tip of the syringe may interfere with the analysis of blood samples. Regarding blood gas analysis, bubbles trapped in a syringe may cause a blood analyzer to produce erroneous results. To obtain accurate results, blood samples need to be mixed thoroughly and all air must be expelled before mixing. Bubbles are typically expelled by tapping the sides of the syringe to force the air or gas to the top of the syringe. Once the bubbles have reached the top of the syringe, a piece of gauze or tissue is put over the top to force the gas (and some blood) out of the syringe. This method poses not only a biohazard risk because the blood sample is freely flowing out of the top of the syringe, but also an exposure risk, as the tip of the syringe is still open to ambient air. A blood analyzer analyzing a blood sample collected in this manner risks producing erroneous results.

Further, blood analyzers often may not permit direct sample input via a syringe or vacuum tube. There, the syringe and/or vacuum tube is merely an intermediate container for the blood sample, and at least a portion of the blood sample must be removed and transferred to a secondary sample container capable of being accommodated by the blood analyzer. Transferring the blood sample to a secondary sample container presents its own exposure risks and may increase obtaining erroneous results.

Clots in a blood sample can also cause erroneous results, which could lead to improper treatment, and cause blockages within the blood analyzer leading to delayed results, damage to the analyzer, or the need to replace consumables, all of which can be costly.

Hemolysis in a blood sample is typically time consuming to detect. Typically, a user would have to centrifuge the sample, and compare the plasma color to a hemolytic index. This requires access to expensive equipment, such as a centrifuge, and takes time. On the other hand, testing without checking for hemolysis can result in lengthier delays, as an unexpected high potassium could place doubt on the entire test result, leading to a treatment delay and increase in cost while a repeat sample is drawn and tested.

Secondary sample containers are often open ended (i.e., lacking a male fitting) and therefore not compatible for use with analyzers in a “hands-free” manner. That is, in prior art systems, a user must either hold the syringe during use with the analyzer (i.e., not hands-free), or the syringe is incompatible and use of the adapter with the analyzer in a hands-free manner of attachment also does not work. Many syringe attachments (e.g., adapters) exist and are compatible with blood gas analyzers. However, these syringe attachments do not couple or attach to the blood analyzer device in a “hands-free” manner, and the operator must remain at the analyzer and hold the syringe while a sample is drawn. If an operator wishes to use the blood gas analyzer in a hands-free manner in prior art systems, the adapter must be removed from the syringe before such that the syringe is directly attached to the analyzer (without the adapter) during use of the analyzer. This work around is sufficient for a de-bubbling adapter, but when using a clot catcher adapter, the benefit of the device is lost if the adapter is removed.

A need exists for an apparatus and method that enables one or more of hemolysis detection, clot removal, hands-free connection of a blood collection device to a blood gas analyzer, and removal of bubbles from a fluid sample. It is to such an apparatus and method that the inventive concepts disclosed and claimed herein are directed.

### SUMMARY OF THE INVENTIVE CONCEPTS

The inventive concepts disclosed and claimed herein generally relate to an apparatus for transferring a fluid sample having a liquid portion and a gas portion from a fluid sample collection apparatus to a liquid sample analyzer. The apparatus includes a barrel and a chromatographic assay assembly. The barrel has a first end, a second end opposite the first end, a sidewall extending between the first end and the second end, an inner surface defining an internal chamber, and an external surface defining at least a portion of a chromatographic assay chamber in fluid communication with the internal chamber via a passage through the external surface of the barrel. Th first end of the barrel has an inlet opening with a clot catcher extending across the inlet opening upstream of the passage and the second end has an outlet opening.

The chromatographic assay assembly is housed in the chromatographic assay chamber and is configured to detect presence of free hemoglobin in the fluid sample. The chromatographic assay assembly includes a sample application pad and a chromatographic detection pad. The sample application pad is configured to receive the fluid sample from the internal chamber. The sample application pad is formed of a first layer of a prefiltration material and a second layer of an asymmetric polysulfone material, wherein the sample application pad is porous to plasma and the free hemoglobin and not porous to red blood cells. The chromatographic detection pad is in fluidic contact with the sample application pad, and the chromatographic detection pad is configured to detect the presence of the free hemoglobin.

In another aspect, the inventive concepts disclosed and claimed herein generally relate to a kit, comprising the apparatus discussed above and a reference device containing a plurality of reference colors. Each reference color corresponds to a different level of hemolysis.

In another aspect, the inventive concepts disclosed and claimed herein generally related to a method of transferring a fluid sample having a liquid portion and a gas portion from a fluid sample collection apparatus to a liquid sample analyzer having a sample probe. The method comprises obtaining an apparatus having a barrel with a first end, a second end, a sidewall extending between the first end and the second end, and an inner surface defining an internal chamber, the first end having an inlet opening with a clot catcher extending across the inlet opening and the second end having an outlet opening. At least a portion the fluid sample is transferred from the fluid sample collection apparatus to the internal chamber of the barrel via the inlet opening so the fluid sample passes through the clot catcher at the inlet opening to capture solids in the fluid sample. A portion of the fluid sample is transferred from the internal chamber of the barrel to a chromatographic assay chamber and a chromatographic assay assembly housed in the chromatographic assay chamber. The chromatographic assay chamber is in fluid communication with the internal chamber downstream of the clot catcher. A presence of free hemoglobin in the fluid sample is detected by the chromatographic assay assembly. The fluid sample is transferred from the internal chamber to the liquid sample analyzer with the sample probe.

### BRIEF DESCRIPTION OF THE DRAWINGS

To assist those of ordinary skill in the relevant art in making and using the inventive concepts disclosed herein, reference is made to the appended drawings and schematics, which are not intended to be drawn to scale, and in which like reference numerals are intended to refer to the same or similar elements for consistency. For purposes of clarity, not every component may be labeled in every drawing. Certain features and certain views of the figures may be shown exaggerated and not to scale or in schematic in the interest of clarity and conciseness. In the drawings:

FIG. 1 is a side perspective view of an exemplary embodiment of an apparatus for removing bubbles according to the inventive concepts disclosed herein shown coupled to a sample receiving assembly.

FIG. 2A is a longitudinal, cross-sectional view of the apparatus of FIG. 1 coupled to a collection syringe illustrating positioning of a filter member and a plunger assembly prior to removal of bubbles from the fluid sample.

FIG. 2B is a longitudinal, cross-sectional view of the apparatus of FIG. 1 coupled to a collection syringe illustrating positioning of the filter member and plunger assembly following removal of bubbles from the fluid sample.

FIG. 2C is a longitudinal, cross-sectional view of the apparatus of FIG. 1 coupled to a collection syringe illustrating the insertion of a probe into the apparatus following removal of bubbles from of the fluid sample.

FIG. 3A is a longitudinal, cross-sectional view of the apparatus of FIG. 1 illustrating a position of the filter member prior to removal of bubbles from the fluid sample.

FIG. 3B is a is a longitudinal, cross-sectional view of the apparatus of FIG. 1 illustrating a position of the filter member following removal of bubbles from the fluid sample.

FIG. 3C is a longitudinal, cross-sectional view of the apparatus of FIG. 1 illustrating the insertion of the probe into the apparatus following removal of bubbles from the fluid sample.

FIG. 4A is a perspective view of the apparatus.

FIG. 4B is a cross-sectional view taken along line 4B-4B of FIG. 4A.

FIG. 5 is an exploded, cross-sectional view of the apparatus.

FIG. 6A is a perspective view of an exemplary embodiment of the filter member according to the inventive concepts disclosed herein.

FIG. 6B is a cross-sectional view taken along line 6B-6B of FIG. 6A.

FIG. 7 is a perspective view of another embodiment of a filter member according to the inventive concepts disclosed herein.

FIG. 8A is a perspective view of another embodiment of a filter member according to the inventive concepts disclosed herein.

FIG. 8B is a cross-sectional view taken along line 8B-8B of FIG. 8A.

FIG. 9 is a perspective view of another exemplary embodiment of an apparatus according to the inventive concepts disclosed herein.

FIG. 10A is a cross-sectional view taken along line 10A-10A of FIG. 9.

FIG. 10B is a cross-sectional view taken along line 10B-10B of FIG. 9.

FIG. 10C is a cross-sectional view taken along line 10C-10C of FIG. 9.

FIG. 10D is a cross-sectional view taken along line 10D-10D of FIG. 9.

FIG. 11 is a longitudinal, cross-sectional view of a portion of the apparatus of FIG. 9 shown coupled to a collection syringe.

FIG. 12 is a partially exploded, perspective view of the apparatus of FIG. 9 illustrated with a chromatographic assay assembly removed.

FIG. 13A is a perspective view of a non-limiting embodiment of a chromatographic assay assembly constructed in accordance with the inventive concepts disclosed herein.

FIG. 13B contains perspective views illustrating a workflow for use of the chromatographic assay assembly of FIG. 13A.

FIG. 14 is a cross-sectional view taken along line 14-14 of FIG. 9.

FIG. 15 schematically depicts one non-limiting embodiment of a reference device utilized with the chromatographic assay assembly constructed in accordance with the present disclosure.

### DETAILED DESCRIPTION OF EXEMPLARY EMBODIMENTS

Before explaining at least one embodiment of the inventive concept(s) in detail by way of exemplary drawings, experimentation, results, and laboratory procedures, it is to be understood that the inventive concept(s) is not limited in its application to the details of construction and the arrangement of the components set forth in the following description or illustrated in the drawings, experimentation and/or results. The inventive concept(s) is capable of other embodiments or of being practiced or carried out in various ways. The language used herein is intended to be given the broadest possible scope and meaning; and the embodiments are meant to be exemplary-not exhaustive. Also, it is to be understood that the phraseology and terminology employed herein is for the purpose of description and should not be regarded as limiting.

Unless otherwise defined, scientific and technical terms used in connection with the presently disclosed and claimed inventive concept(s) shall have the meanings commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All the articles, compositions and/or methods disclosed and claimed herein can be made and executed without undue experimentation, given the present disclosure. While the articles, compositions and methods of the inventive concept(s) have been described in terms of preferred embodiments, it will be apparent to those of skill in the art that variations may be applied to the articles, compositions and/or methods and in the steps or in the sequence of steps of the methods described herein without departing from the concept, spirit and scope of the inventive concept(s). All such similar substitutes and modifications apparent to those skilled in the art are deemed to be within the spirit, scope and concept of the inventive concept(s) as defined by the appended claims.

As utilized under the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the inherent variation of error for the device, the method being employed to determine the value, or the variation that exists among the study subjects.

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, the term “sample” and variations thereof is intended to include biological tissues, biological fluids, chemical fluids, chemical substances, suspensions, solutions, slurries, mixtures, agglomerations, tinctures, slides, powders, or other preparations of biological tissues or fluids, synthetic analogs to biological tissues or fluids, bacterial cells (prokaryotic or eukaryotic), viruses, single-celled organisms, lysed biological cells, fixed biological cells, fixed biological tissues, cell cultures, tissue cultures, genetically engineered cells and tissues, genetically engineered organisms, and combinations thereof, for example.

In the following detailed description of embodiments of the inventive concept, numerous specific details are set forth in order to provide a more thorough understanding of the inventive concept. However, it will be apparent to one of ordinary skill in the art that the inventive concept within the disclosure may be practiced without these specific details. In other instances, well-known features have not been described in detail to avoid unnecessarily complicating the instant disclosure.

Finally, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Described herein, and shown in the accompanying figures, are several non-limiting embodiments of apparatus of the presently claimed and disclosed inventive concepts which may be used in association with collection syringes and liquid sample analyzers for removing bubbles of air or other gases from a fluid sample for analysis by a liquid sample analyzer. The fluid sample is generally from a biological source. A “fluid” refers to any substance that has no fixed shape and yields easily to external pressure.

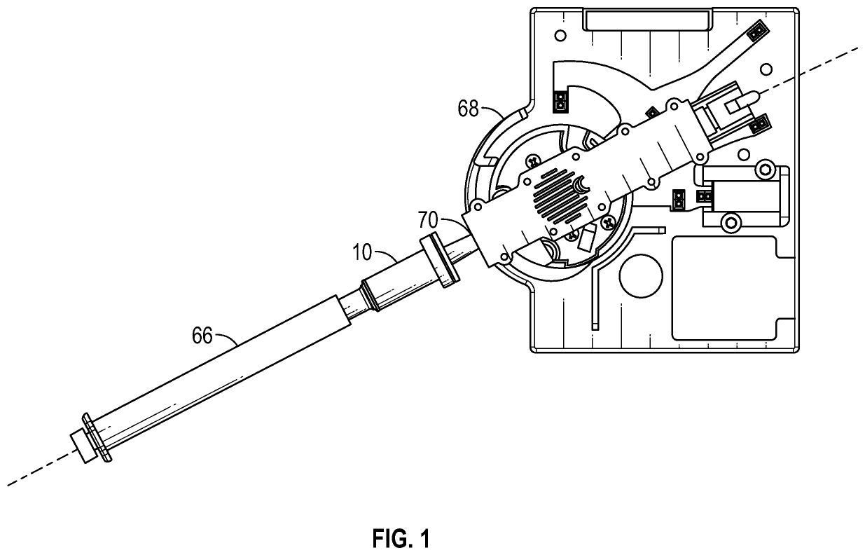

Referring now to the drawings, and more particularly to FIGS. 1-5, shown therein is an exemplary embodiment of an apparatus 10 for transferring a fluid sample from a liquid sample collection apparatus to a liquid sample analyzer and for removing bubbles from the fluid sample constructed in accordance with the inventive concepts disclosed and claimed herein. The apparatus 10 includes a barrel 12, a nozzle cap 14, and a filter member 16.

The barrel 12 includes a first end 18, a second end 20, a sidewall 22, and an inner surface 24. The barrel 12 may be of any suitable size and shape, and formed of any suitable material, such as, without limitation, plastics such as polycarbonate, polystyrene, polyacrylates, and polyurethane, or medical-grade polymers. The sidewall 22 of the barrel 12 extends between the first end 18 and the second end 20 of the barrel 12. The inner surface 24 of the barrel 12 defines an internal chamber 26. The first end 18 has an inlet opening 28 and the second end 20 has an outlet opening 30.

The internal chamber 26 may be of any suitable size and shape to contain a fluid sample 32. The fluid sample 32 may be a liquid biological sample, for example, blood, serum, plasma, or other bodily fluids. The fluid sample 32 may contain a gas portion and a liquid portion. The gas portion of the fluid sample 32 may be, for example, air or other gases. A portion of the gas portion may form bubbles in the fluid sample.

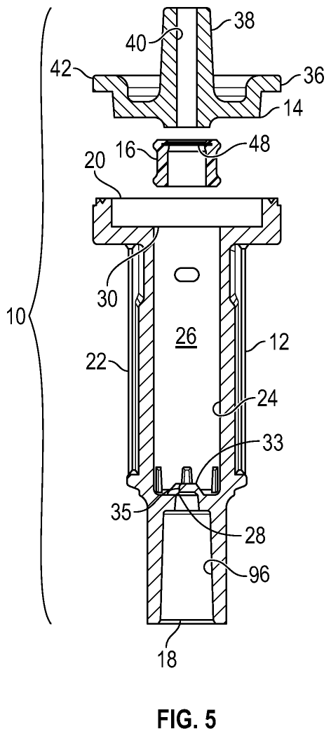

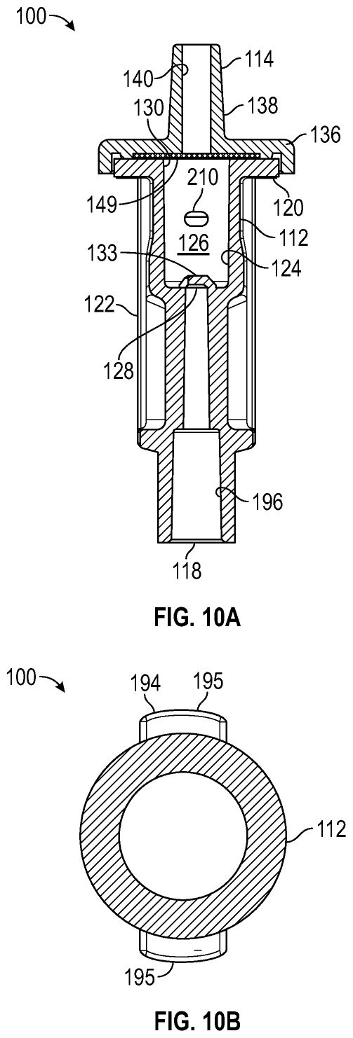

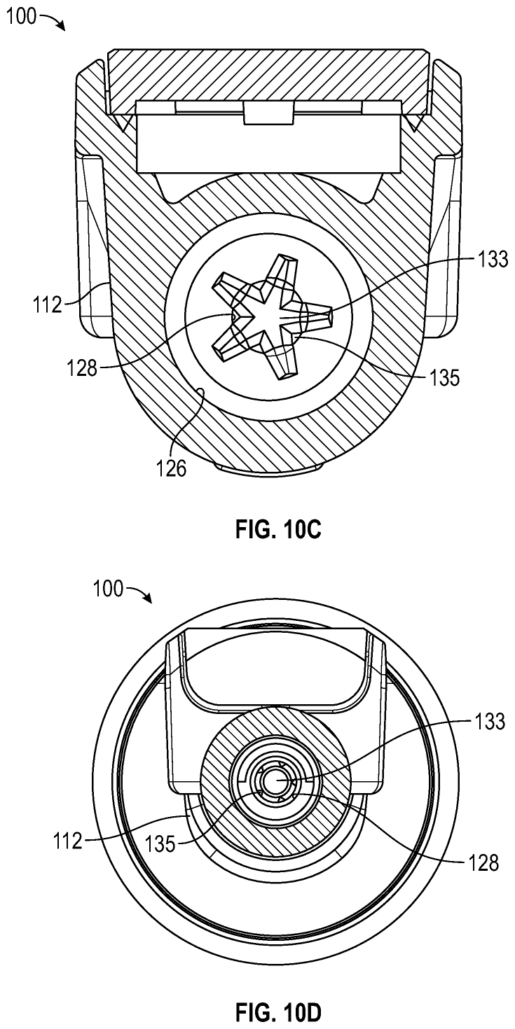

The inlet opening 28 and the outlet opening 30 may have a cross-section of any suitable geometry, including, but not limited to, circular, oval, square, or rectangular. The inlet opening 28 and the outlet opening 30 may be molded or cut into the barrel 12, or otherwise pre-fabricated. The inlet opening 28 may be formed to capture clots as the fluid sample 32 is passed into the internal chamber 26 via the inlet opening 28. The barrel 12 may include a clot catcher 33 (FIG. 5) disposed across the inlet opening 28 so as to define a plurality of apertures 35 that are sized and shaped to allow fluid to pass through into the internal chamber 26, but to catch or prevent solids (i.e., clots) that are larger than a predetermined size to pass through into the internal chamber 26. The solids that may be caught by the clot catcher 33 and prevented from flowing into the internal chamber 26 may include clots and other solids present in the fluid sample 32 that have the predetermined size of, for example, at least about 0.17+/−0.05 mm in diameter or larger. In one non-limiting embodiment, the clot catcher 33 is star-shaped (e.g., as illustrated in FIGS. 10C and 10D as clot catcher 133) so as to cooperate with the inlet opening 28 to define five apertures 35 (only one of which is numbered in FIG. 5) through which fluid is passed through into the internal chamber 26. In this non-limiting embodiment, the five apertures 35 are formed in between five arms of the star-shaped clot catcher 33, where the size and shape of the arms may act as catching elements to catch and prevent clots from passing into the internal chamber 26 (e.g., as shown in FIG. 10C as clot catcher 133 with apertures 135).

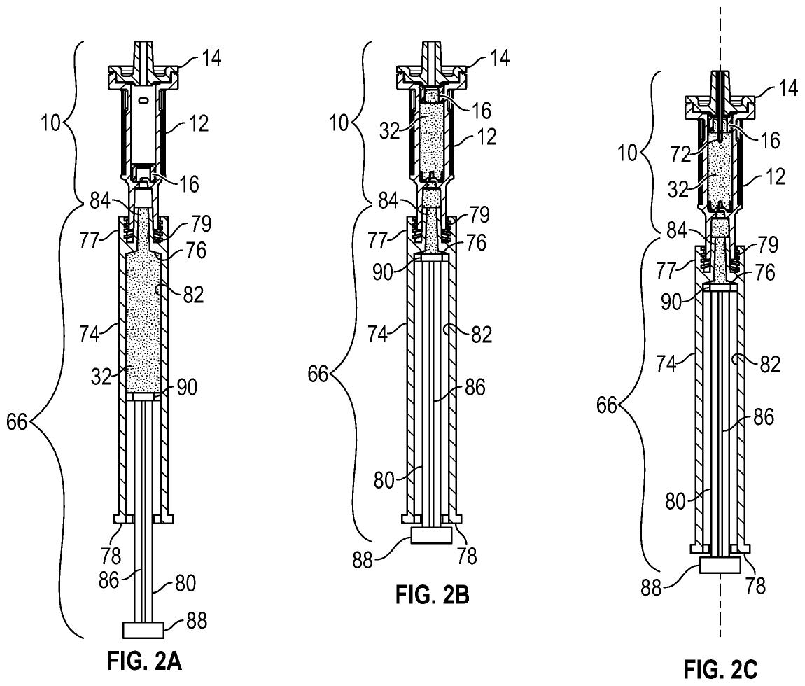

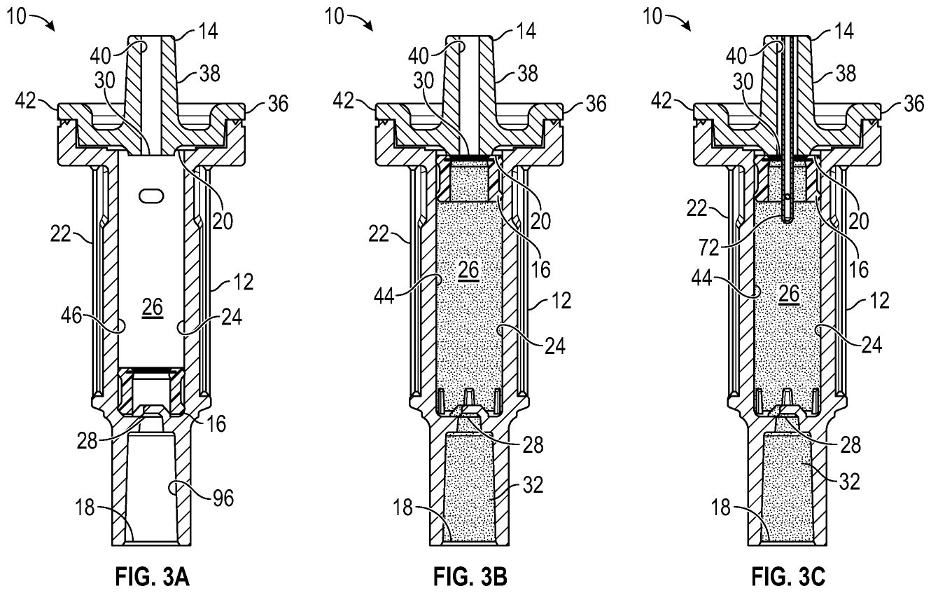

The outlet opening 30 may be provided with a nozzle cap 14. The nozzle cap 14 includes an annular wall 36 and a tubular portion 38 having a bore 40 extending therethrough. The tubular portion 38 may be in the form of a male luer for frictional engagement with a portion of a fluid analyzer 68 (FIG. 1). The liquid sample analyzer 68 includes a sample input port 70 for frictionally receiving the tubular portion 38 and a sample probe 72 (FIGS. 2C and 3C). The sample probe 72 may be axially slidable relative to the sample input port 70. The bore 40 may have a cross-section of any suitable geometry, including, but not limited to, circular, oval, square, or rectangular. The bore 40 may be sized to have a diameter adapted to slidably axially receive a sample probe. The base of the tubular portion 38 flares outwardly and merges with the annular wall 36 at a rim 42, the annular wall 36 tapers downwardly to form an inverted frusto-conical section. The nozzle cap 14 may be releasably coupled to the outlet opening 30 such that the bore 40 is aligned with the outlet opening 30 to permit fluid communication with the internal chamber 26.

The filter member 16 is disposed within the internal chamber 26 so the filter member 16 defines an inlet side 44 and an outlet side 46 of the internal chamber 26. The filter member 16 is positionable between the first end 18 and the second end 20 of the barrel 12. The filter member 16 includes at least one gas-permeable, liquid-impermeable membrane 48. The filter member 16 may be any suitable shape and size to sealingly engage the inner surface 24 of the barrel 12. The filter member 16 may be formed of any suitable material, such as, without limitation, a rubber, an elastomer, a polyolefin-based resin, a fluorine-based resin, or a polyester-based resin. The elastomer may include, for example, a polyvinyl chloride-based elastomer, a polyolefin-based elastomer, a styrene-based elastomer, a polyester-based elastomer, a polyamide-based elastomer, a polyurethane-based elastomer, or a mixture thereof.

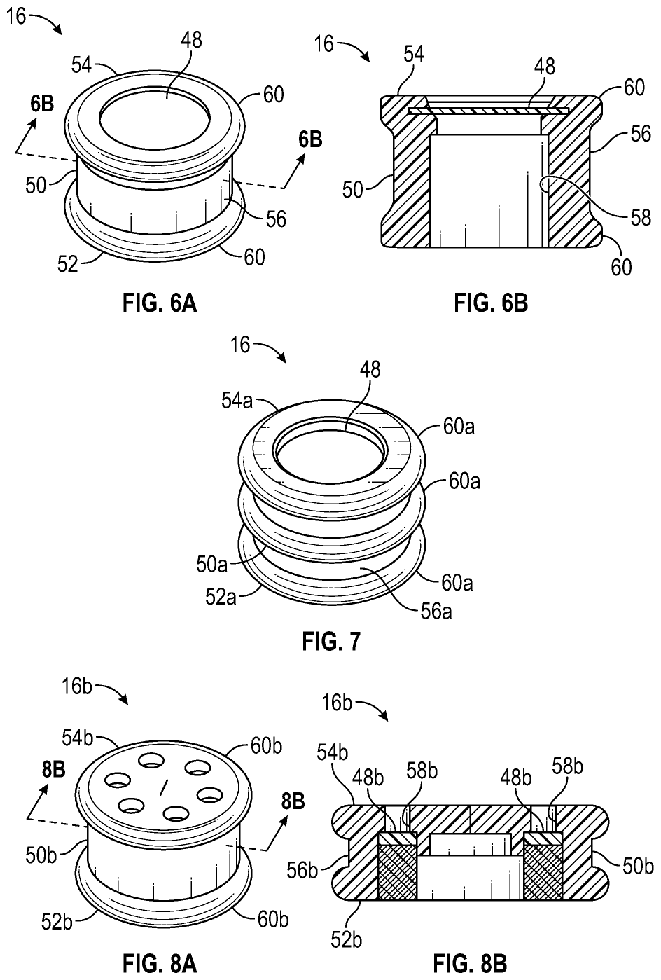

Referring now to FIGS. 6A and 6B, shown therein is a perspective and cross-sectional view, respectively, of an exemplary embodiment of the filter member 16. The filter member 16 comprises a body 50 having a first end 52, a second end 54, and a sidewall 56 extending from the first end 52 to the second end 54. The sidewall 56 of the body 50 defines a passageway 58 extending through the body 50 from the first end 52 to the second end 54. The sidewall 56 of the body 50 may have at least two annular projections 60 extending radially outwardly in slidable, sealing contact with the inner surface 24 of the barrel 12. As shown in FIGS. 6A and 6B, the body 50 may have two annular projections 60 spaced apart from each other. The annular projections 60 may include convex or concave features.

The filter member 16 further includes a gas-permeable, liquid-impermeable membrane 48 which extends across the entirety of the passageway 58. In one embodiment, the gas-permeable, liquid-impermeable membrane 48 is secured to the body 50 adjacent the second end 54 of the body 50, as shown in FIG. 6B.

Referring now to FIG. 7, shown therein is a perspective view of another embodiment of a filter member 16a constructed in accordance with the inventive concepts disclosed and claimed herein. Similar to previously described embodiments, the filter member 16a comprises a body 50a having a first end 52a, a second end 54a, and a sidewall 56a extending from the first end 52a to the second end 54a. The sidewall 56a of the body 50a defines a passageway 58a (not shown) extending through the body 50a from the first end 52a to the second end 54a. As in FIGS. 6A and 6B of the previously described embodiment, the body 50a may have at least two annular projections 60b extending radially outwardly in slidable, sealing contact with the inner surface 24 of the barrel 12. As shown in FIG. 7, the body 50a may have three annular projections 60a spaced apart from each other. The filter member 16a further includes the gas-permeable, liquid-impermeable membrane 48 which extends across the entirety of the passageway 58a. In this embodiment, the gas-permeable, liquid-impermeable membrane 48 is secured to the body 50a adjacent the second end 54a of the body.

Referring now to FIGS. 8A and 8B, shown therein is a perspective and cross-sectional view, respectively, of another embodiment of a filter member 16b. The filter member 16b comprises a body 50b having a first end 52b, a second end 54b, and a sidewall 56b extending from the first end 52b to the second end 54b. Similar to previously described embodiments, the body 50b may have at least two annular projections 50b extending radially outwardly in slidable, sealing contact with the inner surface 24 of the barrel 12. The sidewall 56b of the body 50b defines a plurality of passageways 58b extending through the body 50b from the first end 52b to the second end 54b. In that embodiment, the plurality of passageways 58b may be in a parallel relationship to one another, as shown in FIG. 8B. Further, the filter member 16b may include a plurality of gas-permeable, liquid-impermeable membranes 48b with at least one of the gas-permeable, liquid-impermeable membranes 48b extending across each of the plurality of passageways 58b of the body 50b. The filter member 16b may further include a plurality of porous filter material 62 positioned between the first end 52b of the body 50b and each of the plurality of gas-permeable, liquid-impermeable membranes 48b to prevent solid particulate from contacting the plurality of gas-permeable, liquid-impermeable membranes 48b, as shown in FIG. 8B.

The at least one gas-permeable, liquid-impermeable membrane 48 may be formed of any suitable material, such as, without limitation, polytetrafluoroethylene, polyvinylidene fluoride, polyvinylchloride, polyolefins like polypropylene, polyethylene, polymethylpentene, polyamides, polysulfones, polyetheretherketones, polycarbonates, and combinations including any of these. In one embodiment, the gas-permeable, liquid-impermeable membrane 48 is formed from a material comprising at least one of polytetrafluorethylene, polypropylene, and polyethylene. The at least one gas-permeable, liquid-impermeable membrane 48 may have a thickness suitable for allowing puncture upon application of a mechanical force. The at least one gas-permeable, liquid-impermeable membrane 48 may permit at least a portion of the gas portion of the fluid sample 32, which forms bubbles, to pass across the filter member 16 from the inlet side 44 to the outlet side 46 of the internal chamber 26. The at least one gas-permeable, liquid-impermeable membrane 48 also provides a fluid-tight seal across the filter member 16 to prevent the liquid portion of the fluid sample 32 from passing from inlet side 44 to the outlet side 46 as the fluid sample 32 is passed into the internal chamber 26 via the inlet opening 28 to separate at least a portion of the gas portion from the liquid portion of the fluid sample 32. The filer member 16 is pierceable so a sample probe 72 may be passed through the filter member 16 from the outlet side 46 to the inlet side 44 to withdraw the liquid portion of the fluid sample 32 from the inlet side 44 of the internal chamber 26.

As shown in FIG. 1, the apparatus 10 may be used in association with a fluid sample collection apparatus, such as a collection syringe 66 and the liquid sample analyzer 68 for transferring a fluid sample from the collection syringe 66 to the liquid sample analyzer 68 and to remove bubbles from the fluid sample 32 having a liquid portion and a gas portion. Although, FIG. 1 shows the apparatus 10 associated with the collection syringe 66 and the liquid sample analyzer 68, those skilled in the art will understand and appreciate that the apparatus 10 may independently be associated with collection devices other than the collection syringe 66, such as, for example, a vacuum tube, and medical devices other than the liquid sample analyzer 68. The liquid sample analyzer 68 may be any suitable fluid testing device, such as, without limitation, microfluidic devices, blood gas analyzers, hematology analyzers, urine chemistry analyzers, and the like.

The liquid sample analyzer 68 includes the sample input port 70 and a sample probe 72 (FIGS. 2C and 3C). The sample input port 70 may be sized (e.g., a female luer) to frictionally receive and detachably secure at least a portion of the nozzle cap 14, as shown in FIG. 1 to permit “hands-free” operation of the liquid sample analyzer 68 in a way that the fluid sample in the apparatus 10 may be drawn into the liquid sample analyzer 68 via the sample probe 72 without a user holding the apparatus 10.

The sample probe 72 may be extended to pass through the filter member 16 from the outlet side 46 of the internal chamber 26 to the inlet side 44 to withdraw at least a portion of the fluid sample 32 into the liquid sample analyzer 68 from the inlet side 44 of the internal chamber 26, as shown in FIGS. 2C and 3C. The sample probe 72 may be of a length compatible with sampling from the inlet side 44 of the internal chamber 26.

The collection syringe 66 includes a syringe body 74 having a front end 76, a rear end 78, and a plunger 80. The syringe body 74 defines a reservoir 82 within which the fluid sample 32 may be contained and later expelled via a dispensing opening 84 positioned at the front end 76 of the syringe body 74. The rear end 78 of the syringe body 74 may be open and provided with a body flange 85 to facilitate the collection and expulsion of the fluid sample 32. The syringe body 74 may be any suitable size or shape for collection of fluid samples, such as, for example, a cylindrical shape. The syringe body 74 may also include a collar 77 formed concentrically with the dispensing opening 84 into a cylindrical shape to surround the dispending opening 84. The collar 77 may include an inner peripheral surface in which a threaded engagement portion 79 is formed for engaging the apparatus 10. The syringe body 74 may be constructed of any suitable material, such as glass or plastic. The syringe body 74 may have an outer diameter adapted to coaxially slide within the first end 18 of the apparatus 10.

The plunger 80 may include a shaft 86 that terminates at one end in a plunger flange 88 to facilitate the collection and expulsion of the fluid sample 32. The shaft 86 may, for example, have a cylindrical shape or a columnar shape, and may have a cross-section of a polygonal shape, such as a square, pentagonal, hexagonal, or cruciform shape. The plunger 80 may further include a plunger seal 90 secured to the shaft 86 opposite the plunger flange 88. The plunger 80 may be removably disposed within the syringe body 74 and may be selectively movable within the reservoir 82. The plunger seal 90 has a diameter that permits the plunger seal 90 to create a fluid-tight seal when positioned within the reservoir 82 such that the liquid sample 32 may not move past the plunger seal 90. Further, the plunger seal 90 prevents ambient air from moving from the rear end 78 of the syringe body 74 in a direction past the plunger seal 90. The plunger 80 may be axially displaced relative to the syringe body 74. Movement of the plunger 80 from the rear end 78 to the front end 76 of the syringe body 74 may cause at least a portion of the fluid sample 32 to be expelled from the reservoir 82 and introduced into the inlet opening 28 of the apparatus 10 via the dispensing opening 84. The plunger 80 may be constructed of any suitable polymeric material known in the art.

To remove the gas portion (i.e., bubbles) of the fluid sample 32 from the liquid portion, the collection syringe 66, containing a volume of the fluid sample 32 within the reservoir 82, is releasably attached to the first end 18 of the barrel 12, as shown in FIG. 2A. As shown in FIGS. 2A-2C, the front end 76 of the syringe body 74 may be interlockingly engaged with the apparatus 10 by way of the threaded engagement portion 79.

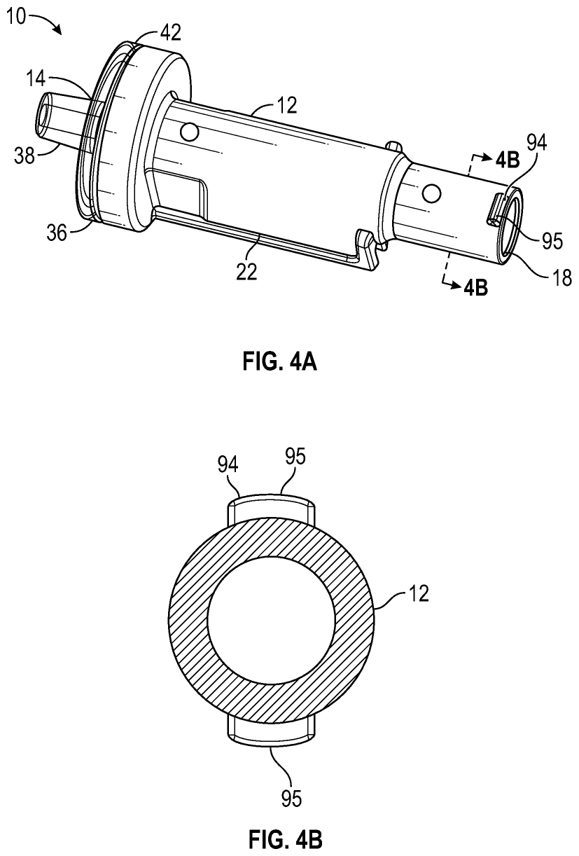

As shown in FIGS. 4A and 4B, the barrel 12 may include a barrel connection portion 94. In one embodiment, the threaded engagement portion 79 of the syringe body 74 is a male luer connection and the barrel connection portion 94 is a female luer connector including in one exemplary embodiment a pair of thread lugs 95 extending radially from the exterior of the barrel 12 and having a thread pitch, size, and geometry corresponding to threaded engagement portion 79 of the syringe body 74. As shown in FIGS. 2A-2C, the threaded engagement portion 79 may interlockingly engage the barrel connection portion 94 such that significant relative movement between the collection syringe 66 and the apparatus 10 is prevented to permit “hands-free” operation of the liquid sample analyzer in a way that the fluid sample from in the collection syringe 66 may be drawn into the liquid sample analyzer via the apparatus 10 without a user holding the collection syringe 66 or the apparatus 10. It will be appreciated that other suitable connectors may be utilized between the apparatus 10 and collection syringe 66, such as a luer slip connection. The first end 18 of the barrel 12 may include a female luer 96 (FIGS. 3A and 5).

In use, the collection syringe 66 and the apparatus 10 are positioned in an upright orientation with the apparatus 10 above the collection syringe 66 and the bubbles in the fluid sample rise to the top of the fluid sample. The plunger 80 of the collection syringe 66 is displaced axially along the reservoir 82 a distance from the rear end 78 towards the front end 76 of the syringe body 74, as shown in FIG. 2A. Movement of the plunger 80 within the reservoir 82 causes at least a portion of the gas portion (i.e., bubbles) of the fluid sample 32 to be expelled from the reservoir 82 and into the internal chamber 26 of the apparatus 10 via the inlet opening 28 and the clot catcher 33, passing through the filter member 16. The gas portion of the fluid sample 32 passes through the filter member 16, and is then ultimately expelled from the internal chamber 26 of the apparatus 10. Once, at least a portion of the gas portion of the fluid sample 32 has been displaced from the reservoir 82, the plunger 80 experiences an initial resistive force.

Upon application of sufficient force to overcome the initial resistive force, the plunger 80 is advanced further into the reservoir 82 towards the front end 76 of the syringe body 74, as shown in FIG. 2B, thereby increasing the internal pressure of the reservoir 82. As the internal pressure of the reservoir 82 increases, it produces a force sufficient to cause at least a portion of the liquid portion of the fluid sample 32 to be expelled from the reservoir 82 and into the internal chamber 26 of the barrel 12 via the inlet opening 28 and the clot catcher 33. The fluid sample 32 entering the internal chamber 26 may cause the filter member 16 to be displaced axially along the internal chamber 26 towards the second end 20 of the barrel 12, as shown in FIGS. 2B and 3B. The filter member 16 may be displaced such that it becomes disposed adjacent the outlet opening 30. This arrangement prevents ambient air from entering the internal chamber 26 and prevents the fluid sample 32 from exiting the internal chamber 26 via the outlet opening 30. In some embodiments, the plunger 80 may be partially extended into the reservoir 82 so less than all of the fluid sample 32 is transferred from the reservoir 82 into the internal chamber 26.

Once the liquid portion of the fluid sample 32 has been expelled from the reservoir 82 and is contained within the internal chamber 26 of the apparatus 10, the sample probe 72 of the liquid sample analyzer 68 may be extended from the sample input port 70 and passed through the filter member 16 to withdraw the liquid portion of the fluid sample 32 from the inlet side 44 of the internal chamber 26, as shown in FIGS. 2C and 3C. In one embodiment, the sample probe 72 pierces the gas-permeable, liquid-impermeable membrane 48 of the filter member 16 to gain fluid access to the inlet side 44 of the internal chamber 26.

After initial insertion of the apparatus 10 into the sample input port 70 by a user, no additional support is required as the fluid sample is drawn into the liquid sample analyzer 68. The connections between the collection syringe 66, the apparatus 10, and the liquid sample analyzer 68 are sufficiently rigid to prevent gravity from tilting down or putting undue stress on the combination of the connected elements such that a hands-free operation can be performed without additional supporting structures to hold the connected elements together in proper alignment. In one non-limiting embodiment and as illustrated in FIGS. 1 and 2C, the connections between the collection syringe 66, the apparatus 10, and the liquid sample analyzer 68 are sufficiently rigid to support the collection syringe 66 and the apparatus 10 in an axially aligned relationship with the sample probe 72 of the liquid sample analyzer 68. As such, the user need not remain at the liquid sample analyzer 68 and need not hold the apparatus 10 and/or the collection syringe 66 while a fluid sample in the apparatus 10 is drawn into the liquid sample analyzer 68 via the sample probe 72.

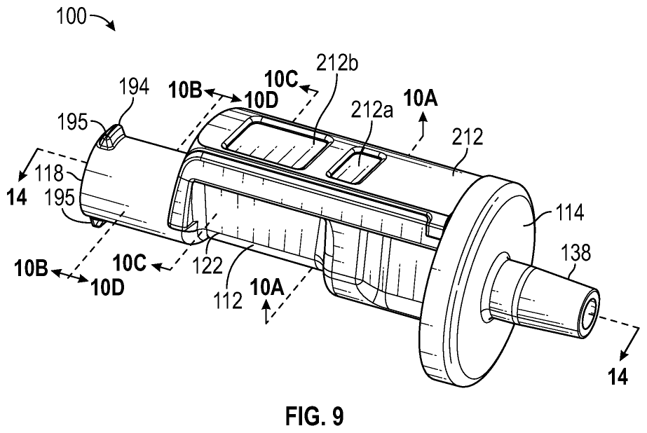

Referring now to FIGS. 9-11, shown is another exemplary embodiment of an apparatus 100 constructed in accordance with the inventive concepts disclosed and claimed herein. The apparatus 100 is similar to the apparatus 10 described above, except as described below. The apparatus 100 includes a barrel 112 and a nozzle cap 114. The apparatus 100 is shown without a filter member 16, which is optional.

The barrel 112 includes a first end 118, a second end 120, a sidewall 122, and an inner surface 124. The barrel 112 may be of any suitable size and shape, and formed of any suitable material, such as, without limitation, plastics such as polycarbonate, polystyrene, polyacrylates, and polyurethane, or medical-grade polymers. The sidewall 122 of the barrel 112 extends between the first end 118 and the second end 120 of the barrel 112. The inner surface 124 of the barrel 112 defines an internal chamber 126. The first end 118 has an inlet opening 128 and the second end 120 has an outlet opening 130.

The internal chamber 126 may be of any suitable size and shape to contain a fluid sample, (e.g., fluid sample 32 shown in FIGS. 2A-2C). The fluid sample may be, for example, blood, serum, plasma, or other bodily fluids. The fluid sample may contain a gas portion and a liquid portion. The gas portion of the fluid sample may be, for example, air or other gases. A portion of the gas portion may form bubbles in the fluid sample.

The inlet opening 128 and the outlet opening 130 may have a cross-section of any suitable geometry, including, but not limited to, circular, oval, square, or rectangular. The inlet opening 128 and the outlet opening 130 may be molded or cut into the barrel 112, or otherwise pre-fabricated. The inlet opening 128 may be formed to capture clots as the fluid sample 32 is passed into the internal chamber 126 via the inlet opening 128. The barrel 112 may include a clot catcher 133 disposed across the inlet opening 28 so as to define a plurality of apertures 135 that are sized and shaped to allow fluid to pass through into the internal chamber 126, but to catch or prevent solids (i.e., clots) that are larger than a predetermined size to pass through into the internal chamber 126. The solids that may be caught by the clot catcher 133 and prevented from flowing into the internal chamber 126 may include clots and other solids present in the fluid sample 32 that have the predetermined size of, for example, at least about 0.17+/−0.05 mm in diameter or larger. In one non-limiting embodiment, the clot catcher 133 is star-shaped (e.g., as illustrated in FIGS. 10C and 10D as clot catcher 133) so as to cooperate with the inlet opening 128 to define five apertures 135 (only one of which is numbered in FIGS. 10C and 10D) through which fluid is passed through into the internal chamber 126. In this non-limiting embodiment, the five apertures 135 are formed in between five arms of the star-shaped clot catcher 133, where the size and shape of the arms may act as catching elements to catch and prevent clots from passing into the internal chamber 126 (e.g., as shown in FIG. 10C as clot catcher 133 with apertures 135).

The outlet opening 130 may be provided with the nozzle cap 114. The nozzle cap 114 includes a cap portion 136 and a tubular portion 138 having a bore 140 extending therethrough. The tubular portion 138 may be in the form of a male luer for frictional engagement with the sample input port 70 of the fluid analyzer 68 (FIG. 1) to permit “hands-free” operation of the liquid sample analyzer 68 in way that the fluid sample in the apparatus 100 may be drawn into the liquid sample analyzer 68 without a user holding the apparatus 100.

The bore 140 may have a cross-section of any suitable geometry, including, but not limited to, circular, oval, square, or rectangular. The bore 140 may be sized to have a diameter adapted to slidably axially receive the sample probe 72. The base of the tubular portion 138 flares outwardly and merges with the cap portion 136. The nozzle cap 114 may be coupled to the second end of the barrel 112 in a suitable manner such that the bore 140 is aligned with the outlet opening 130 to permit fluid communication with the internal chamber 126.

A gas-permeable, liquid impermeable membrane 149 (FIG. 10A) may be secured to the barrel 112 adjacent the second end 120 of the barrel 112 to provide a fluid-tight seal across the outlet opening 130 to prevent the liquid portion of the fluid sample from passing into the outlet opening 130 from the internal chamber 126. The gas-permeable, liquid impermeable membrane 149 is pierceable so the sample probe 72 may be passed through the gas-permeable, liquid impermeable membrane 149 to withdraw the liquid portion of the fluid sample from the internal chamber 126.

The gas-permeable, liquid-impermeable membrane 149 may be formed of any suitable material, such as, without limitation, polytetrafluoroethylene, polyvinylidene fluoride, polyvinylchloride, polyolefins like polypropylene, polyethylene, polymethylpentene, polyamides, polysulfones, polyetheretherketones, polycarbonates, and combinations including any of these. In one embodiment, the gas-permeable, liquid-impermeable membrane 149 is formed from a material comprising at least one of polytetrafluorethylene, polypropylene, and polyethylene. The gas-permeable, liquid-impermeable membrane 149 may have a thickness suitable for allowing puncture upon application of a mechanical force.

Similar to the apparatus 10, the apparatus 100 may be used in association with the collection syringe 66 and the liquid sample analyzer 68.

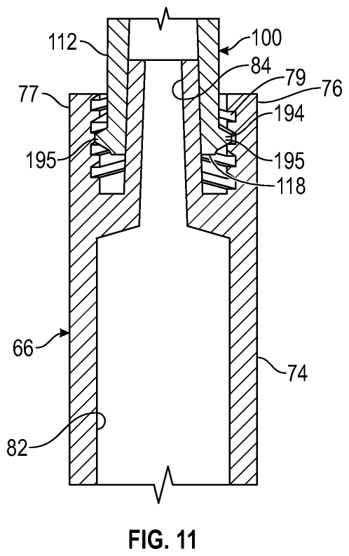

To establish fluid communication between the collection syringe 66 and the apparatus 100, the collection syringe 66, containing a volume of the fluid sample within the reservoir 82, may be interlockingly engaged with the first end 118 of the barrel 112. As shown in FIG. 11, the front end 76 of the syringe body 74 may be interlockingly engaged with the apparatus 100 by way of the threaded engagement portion 79. As shown in FIGS. 9 and 10B, the barrel 112 may include a barrel connection portion 194. In one embodiment, the threaded engagement portion 79 of the syringe body 74 is a male luer connector and the barrel connection portion 194 is a female luer connector including in one exemplary embodiment a pair of thread lugs 195 extending radially from the exterior of the barrel 112 and having a thread pitch, size, and geometry corresponding to threaded engagement portion 79 of the syringe body 74. As shown in FIG. 11, the threaded engagement portion 79 may interlockingly engage the barrel connection portion 194 such that significant relative movement between the collection syringe 66 and the apparatus 100 is prevented to permit “hands-free” operation of the liquid sample analyzer 68 in way that the fluid sample from in the collection syringe 66 may be drawn into the liquid sample analyzer 68 via the apparatus 100 without a user holding the collection syringe 66 or the apparatus 100. It will be appreciated that other suitable connectors may be utilized between the apparatus 100 and the collection syringe 66, such as a luer slip connection. The first end 118 of the barrel 112 may include a female luer 196 (FIG. 10A).

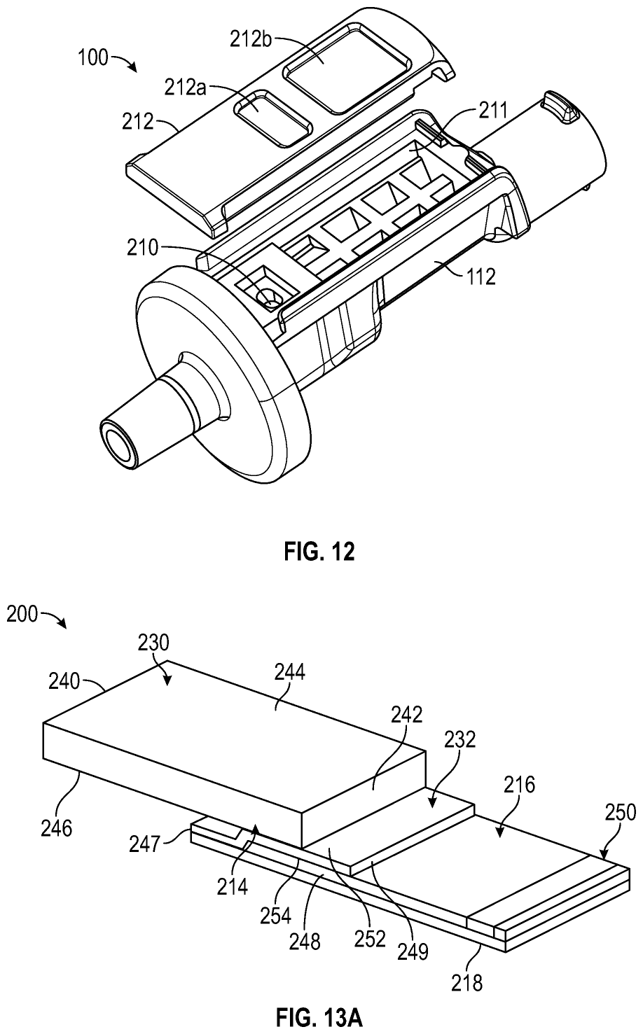

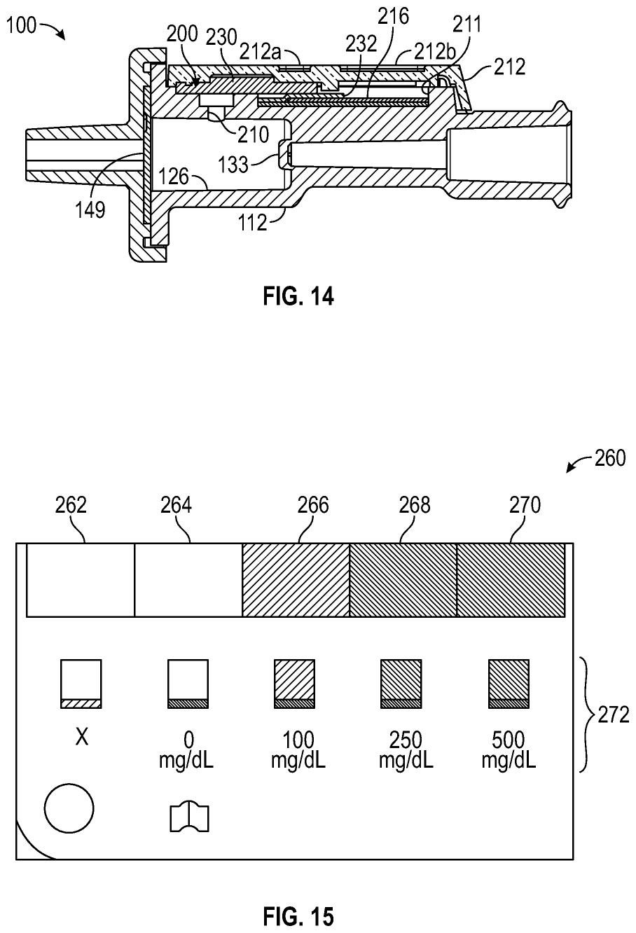

Referring now to FIGS. 12-15, the apparatus 100 may further include a chromatographic assay assembly 200 for detecting free hemoglobin in a fluid sample, such as the fluid sample 32. The chromatographic assay assembly 200 is housed in a chromatographic assay chamber 211 (FIGS. 12 and 14) of the barrel 112. In one non-limiting embodiment, the chromatographic assay chamber 211 may be formed in part by a portion of an exterior surface of the sidewall 122 of the barrel 112 and a cover 212. The chamber 211 is in fluid communication with the internal chamber 126 of the barrel 112 via a passage 210 downstream of the clot catcher 133 and upstream of the membrane 149, as shown in FIG. 14. The chromatographic assay chamber 211 is configured to hold the chromatographic assay assembly 200 so at least a portion of the fluid sample in the internal chamber 126 of the barrel 112 passes into the chromatographic assay chamber 211 and into contact with the chromatographic assay assembly 200.

The cover 212 may be formed entirely or in part with the same material as that forming the barrel 112. For example, the cover 212 may be formed of any suitable material, such as, without limitation, plastics such as polycarbonate, polystyrene, polyacrylates, and polyurethane, or medical-grade polymers. The cover 212 may be transparent or formed with transparent windows for viewing the chromatographic assay assembly 200. In a manner to be described below, the cover 212 may include a fill panel 212a for observing when a sufficient volume of the fluid sample 32 has entered the chamber 211 and a read panel 212b for assessing the hemolysis level, as shown in FIGS. 12 and 14. In one non-limiting embodiment, the fill pane 212a and the read pane 212b may be delineated from the remainder of the cover 212 by reducing the thickness of the cover 212 so as to define the fill pane 212a and the read pane 212b. In another embodiment, the fill pane 212a and the read pane 212b may be formed of a transparent material that is different from the material used to form the cover 212, such as glass. In another embodiment, the fill pane 212a and the read pane 212b may be voids or openings through the cover 212.

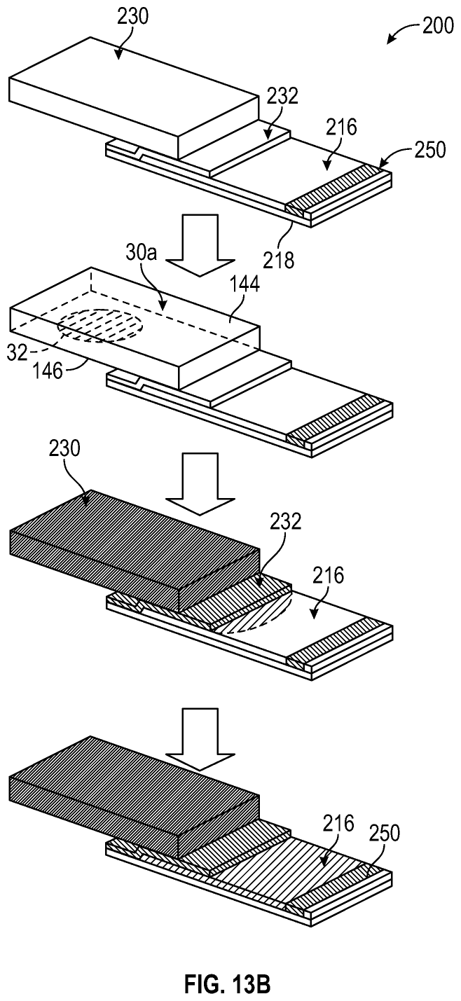

As shown in FIGS. 13A, 13B, and 14, the chromatographic assay assembly 200 includes a sample application pad 214 in fluidic contact with a chromatographic detection pad 216. The sample application pad 214 is configured for application of a portion of the fluid sample 32 to the chromatographic assay assembly 200. The sample application pad 214 may receive and absorb (a portion of) the fluid sample 32, and the fluid sample 32 from the internal chamber 126 via the passage 210 may then be absorbed into the chromatographic detection pad 216 from the sample application pad 214.

Referring to FIG. 13A, the sample application pad 214 may be formed of two layers with different sizes and dimensions and do not fully overlap with one another. The sample application pad 214 includes a first layer 230 formed of a prefiltration material, such as a glass fiber material, and a second layer 232 formed of a different filtration material, such as an asymmetric polysulfone material. The first layer 230 has a first end 240, a second end 242, an upper surface 244, and a lower surface 246. The second layer 232 has a first end 247, a second end 249, an upper surface 252, and a lower surface 254. At least a portion of the first layer 230 adjacent to the second end 242 thereof overlaps a portion of the second layer 232 between the first and second ends 247 and 249 thereof. The overlapping portions of the first and second layers 230 and 232 may be attached to one another, or the overlapping portion of the first layer 230 may simply be layered upon the second layer 232, so a portion of the lower surface 246 of the first layer 230 is in contact with a portion of the upper surface 252 of the second layer 232.

The lower surface 246 of the first layer 230 is aligned and is in fluid communication with the passage 210 and positioned to receive the fluid sample 32 from the internal chamber 126 of the barrel 112.

Accordingly, the first layer 230 and the second layer 232 of the sample application pad 214 may partially overlap with one another to form an overlapping portion and a non-overlapping portion. Referring to the workflow shown in FIG. 13B of the chromatographic assay assembly 200, the non-overlapping portion of the first layer 230 (at the lower surface 246) may receive and absorb a portion of the fluid sample 32 from the internal chamber 126 via the passage 210 (shown in the second panel of FIG. 13B). The fluid sample 32 may then be absorbed into the chromatographic detection pad 216 from the overlapping portion of the sample application pad 214 (shown in the third and fourths panels of FIG. 13B). In particular, as the fluid sample 32 is absorbed or saturated throughout the first layer 230 (which may be visible through fill pane 212a shown in FIG. 12), the fluid sample 32 may then be filtered or passed through from the overlapping portion of the first layer 230 (at the lower surface 246) to the overlapping portion of the second layer 232 (at the upper surface 252), as shown in the third panel of FIG. 13B. As the fluid sample 32 is absorbed or saturated throughout the second layer 232, the fluid sample 32 may then be filtered or passed through from second layer 232 (at the lower surface 254) to a sample application site 248 of the chromatographic detection pad 216 (at a portion of its upper surface that overlaps with the lower surface 254 of the second layer 232), as shown in the third and fourth panels of FIG. 13B. The component(s) of the fluid sample 32 (i.e., plasma and free hemoglobin, if any) absorbed by the chromatographic detection pad 216 then flow via capillary action from the sample application site 248 to a detection site 250 on the chromatographic detection pad 216 for detection of free hemoglobin indicative of hemolysis (shown in the fourth panel).

The sample application pad 214 is porous to plasma and free hemoglobin present in the fluid sample 32 but is not porous to red blood cells so red blood cells present in the fluid sample 32 are retained within the two layers 230 and 232 of the sample application pad 214 and are thereby prevented from flowing there through to the chromatographic detection pad 216. Thus, the sample application pad 214 acts as a filter to filter the fluid sample 32 received through the passage 210 such that red blood cells present in the fluid sample 32 (received from the internal chamber 126 via the passage 210) are filtered out and retained within the two layers 230 and 232, while plasma and free hemoglobin present in the fluid sample 32 may pass through the pores of the sample application pad 214 and be received by and absorbed into the chromatographic detection pad 216.

The multi-layered or dual-layered sample application pad 214 advantageously provides improved removal or filtration of red blood cells and detection of hemolysis (via detecting the presence of free hemoglobin on the chromatographic detection pad 216) because, for example, the first layer 230 of the sample application pad 214 may be capable of retaining at least a portion of the red blood cells and other larger cellular components (without lysing the cells) in the fluid sample 32 and thereby reducing the amount of red blood cells and other larger cellular components that flow into the second layer 232 of the sample application pad 214 so as to not overburden filtration occurring at the second layer 232. In particular, but not by way of limitation, the first and/or second layers 230, 232 have varying pore sizes that are reduced in size when moving from the first layer 230 to the lower surface 254 of the second layer 232 (i.e., moving in the direction toward the chromatographic detection pad).

The chromatographic detection pad 216 defines a path for capillary fluid flow. Components of the fluid sample 32 capable of flowing through the sample application pad 214 then flow through the chromatographic detection pad 216 by capillary action (which may also be referred to as capillary flow). The chromatographic detection pad 216 has a first end portion and a second end portion. The chromatographic detection pad 216 may be made of any suitable material that allows plasma and free hemoglobin from the fluid sample 32 to freely flow therethrough by capillary action. As one non-limiting example, the chromatographic detection pad 216 may be a nitrocellulose membrane. The chromatographic detection pad 216 may have pores through which certain components of the fluid sample 32 moves by capillary action. The majority of the pores of the chromatographic detection pad 216 may all be substantially the same size or fall within a range of values.

Referring to FIG. 13A, the first end portion of the chromatographic detection pad 216 is in fluidic contact with the lower surface 254 of the second layer 232 of sample application pad 214 and forms a sample application site 248 on the chromatographic detection pad 216. As shown in FIGS. 13A-13B, the chromatographic detection pad 216 also has a detection site 250 spaced apart from (and, in certain non-limiting embodiments, downstream of) the first end portion/sample application site 248, with the detection site 250 being disposed between the first and second end portions or substantially adjacent to or closer to the second end portion than the first end portion.

The sample application pad 214 only covers a portion of the chromatographic detection pad 216 adjacent the first end portion and sample application site 248 thereof, but not the detection site 250 thereof; in this manner, the flow of sample through the chromatographic detection pad 216 into the detection site 250 thereof is visible via the read pane 212b of the cover 212 (as shown in FIGS. 12 and 14).

Referring to FIG. 13A, the chromatographic assay assembly 200 may further include a backing material 218 to which a lower surface of the chromatographic detection pad 216 is attached or otherwise associated (such as, but not limited to, via double stick adhesive).

When a fluid sample 32 (such as, but not limited to, a whole blood sample, urine, or other red blood cell-containing liquid sample) is applied to the chromatographic assay assembly 200, free hemoglobin flows through the sample application pad 214 into the chromatographic detection pad 216, and then from the sample application site 248 of the chromatographic detection pad 216 to the detection site 250 thereof. In this manner, free hemoglobin (indicative of hemolysis) is detectable at the detection site 250 via a color change due to the red color of free hemoglobin. The chromatographic detection pad 216 may be formed of a material that is white in color, thus allowing for a visual read or detection of hemolysis via color change of the chromatographic detection pad 216 at the detection site 250 via the read pane 212b of the cover 212.

While one particular, non-limiting embodiment of a chromatographic assay assembly 200 is shown in FIGS. 13A and 13B, it will be understood that the design and configuration of the chromatographic assay assembly 200 shown is for purposes of example only. The scope of the present disclosure includes adapting the design and configuration of the chromatographic assay assemblies of the present disclosure, so long as the chromatographic assay assembly remains capable of functioning in accordance with the present disclosure.

For example (but not by way of limitation), it will be understood that the first layer 230 (of prefiltration material) and the second layer 232 (of asymmetric polysulfone material) of the sample application pad 214 need not be symmetrical with one another (i.e., they can differ from one another in size, length, width, and/or thickness). In addition, the first and second layers 230, 232 of the sample application pad 214 need not be congruous with one another, and therefore each layer can have an area that does not overlap with the other layer. The only requirement is that at least a portion of the first layer 230 must overlap a sufficient portion of the second layer 232 so the sample can flow through the first layer 230 into the second layer 232 and then flow from the second layer 232 into the sample application site 248 of the chromatographic detection pad 216.

In use, the collection syringe 66 is interlockingly engaged with the apparatus 100 and both are positioned in an upright orientation with the apparatus 100 above the collection syringe 66. When the apparatus 100 includes no filter member 16, a user may remove bubbles from the fluid sample in a conventional manner discussed above. The plunger 80 of the collection syringe 66 is displaced axially along the reservoir 82 a distance from the rear end 78 towards the front end 76 of the syringe body 74. Movement of the plunger 80 within the reservoir 82 causes at least a portion of the fluid sample to be expelled from the reservoir 82 and into the internal chamber 126 of the apparatus 100 via the inlet opening 128 and through the clot catcher 133. Any gas portion of the fluid sample passes through the gas-permeable, liquid-impermeable membrane 149.

A portion of the fluid sample passes into the chromatographic assay chamber 211 via the passage 210 and into contact with the sample application pad 214. FIG. 13B illustrates a workflow for the chromatographic assay assembly 200. The fluid sample 32 (e.g., blood sample) is applied to the lower surface 246 of the first layer 230 of the sample application pad 214 and enters the first layer 230, as shown in the second panel of FIG. 13B. The fluid sample 32 saturates the first layer 230 and flows through the overlapping portion into the second layer 222 (third panel). Saturation of the first layer 230 may be determined by viewing the first layer 230 through the fill pane 212a of the cover 212, which is aligned with at least a portion of the sample application pad 214.

When the first layer 230 is saturated, plasma and free hemoglobin (if any) from the blood sample 32 then passes through the second layer 232 and enters the chromatographic detection pad 216 and flows from the sample application site 248 to the detection site 250 thereof for detection of any hemolysis present (fourth panel). A control line may be present on the chromatographic detection pad 216 at or near the detection site 250. In one non-limiting embodiment, the plasma that reaches the detection site 250 turns the control line from yellow to blue, indicating that the chromatographic assay assembly 200 is ready to be read through the read pane 212b, which is aligned with the detection site 250.

FIG. 15 illustrates one non-limiting embodiment of a reference device 260 that can be utilized with a chromatographic assay assembly 200 to visually determine the level of hemolysis in a fluid sample 32. The reference device 260 contains a plurality of reference colors (such as, but not limited to, the reference colors 262, 264, 266, 268, and 270, with color 262 having the white/default color of the chromatographic detection pad 16 and serving as a negative control, and colors 264, 266, 268, and 270 being various shades of pink/red in increasing intensities/hues, wherein the darker intensities/hues correlate to higher amounts/degrees of hemolysis); five reference colors shown for purposes of illustration only). In addition, the reference device 260 also contains a key 272 that correlates each of the reference colors 262, 264, 266, 268, and 270 to a specific concentration of free hemoglobin. That is (and for purposes of example only), color 262 of the key 272 is the negative control, while color 264 indicates that 0 mg/dL free hemoglobin is present, color 266 indicates that 100 mg/dL free hemoglobin is present, color 268 indicates that 250 mg/dL free hemoglobin is present, and color 270 indicates that 500 mg/dL free hemoglobin is present. In this manner, an individual can determine a level of hemolysis in a liquid biological sample in any setting (including, but not limited to, point-of-care or in-home settings) by comparing the color at the detection site 250 to the reference colors 264-270 of the reference device 260.

The design and configuration of the reference device 260 of FIG. 15 is shown for purposes of example only; it will be understood that the reference device 260 may be provided with less than five reference colors or more than five reference colors thereon (such as, but not limited to, two, three, four, five, six, seven, eight, nine, ten, or more reference colors thereon). In addition, the shapes and placement of the reference colors may be different. Also, the key 272 may be provided with different shapes/placement that differs from that shown in FIG. 15. The design and configuration of each of the components of the reference device 260 (such as, but not limited to, the reference colors and the key 272) may easily be adapted by a person of ordinary skill in the art to possess any design and configuration that will allow the reference device 260 to function in accordance with the present disclosure. Alternatively, a medical diagnostics device may be utilized to optically detect the level of hemolysis in a fluid sample, such as optically detecting the level of hemolysis through read pane 212b, where the medical diagnostics device includes an optical sensor, a processor, and a light source directed at the detection site 250. The medical diagnostics device may be the liquid sample analyzer 68 or a different device.

A method of optically testing a fluid sample for hemolysis may include measuring the characteristics of the light reflected by the detection site 250 of the chromatographic assay assembly 200, as described above, after a portion of the liquid sample 32 has been applied to the sample application pad 214 and free hemoglobin has flowed through the sample application pad 214 and into the chromatographic detection pad 216 from the sample application site 248 to the detection site 250. The measured amount(s) of, for example, red, orange, green, and/or blue light can then be used in determining the level of free hemoglobin by, for example, comparing the measured amount(s) against one or more reference values. In exemplary embodiments, the method of testing a fluid sample for hemolysis may be performed optically by a medical diagnostic device (not shown) or visually by a medical provider. A medical provider may, for example, visually compare the completed chromatographic assay assembly 200 against a reference device (such as, but not limited to, the reference device 260 shown in FIG. 15), wherein the reference device contains a plurality of reference colors which each correspond to a different level of hemolysis, to visually determine the hemolysis of the liquid sample 32.

This method may detect the levels of hemoglobin that exceed a predetermined interference value (for example, a manufacturers' interference level). If the sample is above the interference value, the sample would be flagged to inform the end user (i.e., the relevant healthcare provider) that the sample is hemolyzed and therefore compromised and should not be used for further testing.

If the level of hemolysis determined in the sample (by visual and/or optical detection of free hemoglobin levels, discussed above) is below a predetermined threshold, then the sample is not compromised and may be subject to further testing. For the uncompromised sample to undergo further testing, the apparatus 100 is engaged with a testing instrument, such as the liquid sample analyzer 68, with the collection syringe 66 engaged with the apparatus 100. The sample probe 72 (FIG. 3C) of the liquid sample analyzer 68 may then be extended from the sample input port 70 and passed through the gas-permeable, liquid-impermeable membrane 149 to withdraw the liquid portion of the fluid sample from the internal chamber 126 in a “hands free” manner without a user holding the collection syringe 66 or the apparatus 100.

After initial insertion of the apparatus 100 into the sample input port 70 by a user, no additional support is required as the fluid sample is drawn into the liquid sample analyzer 68. The connections between the collection syringe 66, the apparatus 100, and the liquid sample analyzer 68 are sufficiently rigid to prevent gravity from tilting down or putting undue stress on the combination of the connected elements such that a hands-free operation can be performed without additional supporting structures to hold the connected elements together in proper alignment. Similar to that illustrated in FIG. 2C in reference to the apparatus 10, the connections between the collection syringe 66, the apparatus 100, and the liquid sample analyzer 68 are sufficiently rigid to support the collection syringe 66 and the apparatus 100 in an axially aligned relationship with the sample probe 72 of the liquid sample analyzer 68. As such, the user need not remain at the liquid sample analyzer 68 and need not hold the apparatus 100 and/or the collection syringe 66 while a fluid sample in the apparatus 100 is drawn into the liquid sample analyzer 68 via the sample probe 72.

From the above description, it is clear that the inventive concept(s) disclosed herein is well adapted to carry out the objects and to attain the advantages mentioned herein as well as those inherent in the inventive concept disclosed herein. While exemplary embodiments of the inventive concept disclosed herein have been described for purposes of this disclosure, it will be understood that numerous changes may be made which will readily suggest themselves to those skilled in the art and which are accomplished without departing from the scope of the inventive concept disclosed herein and defined by the appended claims.

The following is a list of non-limiting illustrative embodiments of the inventive concepts disclosed herein:

Illustrative embodiment 1. An illustrative apparatus for transferring a fluid sample having a liquid portion and a gas portion from a fluid sample collection apparatus to a liquid sample analyzer, comprising:
a barrel having a first end, a second end opposite the first end, a sidewall extending between the first end and the second end, an inner surface defining an internal chamber, and an external surface defining at least a portion of a chromatographic assay chamber in fluid communication with the internal chamber via a passage through the external surface of the barrel, the first end having an inlet opening having a clot catcher extending across the inlet opening upstream of the passage and the second end having an outlet opening; and
a chromatographic assay assembly housed in the chromatographic assay chamber configured to detect presence of free hemoglobin in the fluid sample, the chromatographic assay assembly comprising:
   a sample application pad configured to receive the fluid sample from the internal chamber, wherein the sample application pad is formed of a first layer of a prefiltration material and a second layer of an asymmetric polysulfone material, wherein the sample application pad is porous to plasma and the free hemoglobin and not porous to red blood cells; and
   a chromatographic detection pad in fluidic contact with the sample application pad, the chromatographic detection pad being configured to detect the presence of the free hemoglobin.

Illustrative embodiment 2. The illustrative apparatus of illustrative embodiment 1, wherein the chromatographic assay chamber is defined by a portion of the outer surface of the barrel and a cover, the cover having a transparent fill pane aligned with the sample application pad for viewing flow of the fluid sample through the sample application pad and a transparent read pane aligned with a detection site of the chromatographic detection pad for viewing the detection site.

Illustrative embodiment 3. The illustrative apparatus of any one of the preceding illustrative embodiments, wherein the first end of the barrel has a barrel connection portion engageable with a portion of the fluid sample collection apparatus, and wherein the second end of the barrel has a tubular portion configured to engage with the liquid sample analyzer so a combination of the fluid sample collection apparatus and the barrel is attachable to the liquid sample analyzer with no additional support.

Illustrative embodiment 4. The illustrative apparatus of any one of the preceding illustrative embodiments, wherein the barrel connection portion comprises at least one thread lug extending radially outwardly from the sidewall of the barrel so as to be interlockingly engageable with a portion of the fluid sample collection apparatus.

Illustrative embodiment 5. The illustrative apparatus of any one of the preceding embodiments, wherein the barrel connection portion comprises a pair of thread lugs extending radially outwardly from the sidewall of the barrel so as to be interlockingly engageable with the fluid sample collection apparatus.

Illustrative embodiment 6. The illustrative apparatus of any one of the preceding illustrative embodiments, wherein the barrel connection portion is a female luer connector.

Illustrative embodiment 7. The illustrative apparatus of any one of the preceding illustrative embodiments, wherein the tubular portion of the barrel is a male luer connector configured to engage with the liquid sample analyzer.

Illustrative embodiment 8. The illustrative apparatus of any one of the preceding illustrative embodiments, further comprising further comprising a gas-permeable, liquid-impermeable membrane secured to the barrel adjacent the second end of the barrel, the gas-permeable, liquid-impermeable membrane being pierceable so a sample probe is configured to be passed through the gas-permeable, liquid-impermeable membrane to the internal chamber.

Illustrative embodiment 9. The illustrative apparatus of any one of the preceding illustrative embodiments, wherein the gas-permeable, liquid-impermeable membrane is formed from a material comprising at least one of polytetrafluorethylene, polypropylene, and polyethylene.

Illustrative embodiment 10. The illustrative apparatus of any one of the preceding illustrative embodiments, further comprising:
a filter member disposed within the internal chamber so the filter member defines an inlet side and an outlet side of the internal chamber and so the filter member is positionable between the first end and the second end of the barrel, the filter member having at least one gas-permeable, liquid-impermeable membrane configured to permit at least a portion of the gas portion of the fluid sample to pass across the filter member from the inlet side to the outlet side of the internal chamber and to provide a fluid-tight seal across the filter member to prevent the liquid portion of the fluid sample from passing from the inlet side to the outlet side as the fluid sample is passed into the internal chamber via the inlet opening to separate at least a portion of the gas portion from the liquid portion of the fluid sample,
wherein the filter member is pierceable so a probe is configured to be passed through the filter member from the outlet side to the inlet side to withdraw the liquid portion of the fluid sample from the inlet side of the internal chamber.

Illustrative embodiment 11. The illustrative combination of any one of the preceding illustrative embodiments, wherein the filter member is slidably disposed in the internal chamber of the barrel.

Illustrative embodiment 12. An illustrative kit, comprising:
the apparatus of any one of the preceding illustrative embodiments; and
a reference device containing a plurality of reference colors, wherein each reference color corresponds to a different level of hemolysis.

Illustrative embodiment 13. An illustrative method of transferring a fluid sample having a liquid portion and a gas portion from a fluid sample collection apparatus to a liquid sample analyzer having a sample probe, the method comprising:
obtaining an apparatus having a barrel with a first end, a second end, a sidewall extending between the first end and the second end, and an inner surface defining an internal chamber, the first end having an inlet opening with a clot catcher extending across the inlet opening and the second end having an outlet opening;
transferring at least a portion the fluid sample from the fluid sample collection apparatus to the internal chamber of the barrel via the inlet opening so the fluid sample passes through the clot catcher at the inlet opening to capture solids in the fluid sample;
passing a portion of the fluid sample from the internal chamber of the barrel to a chromatographic assay chamber and a chromatographic assay assembly housed in the chromatographic assay chamber, the chromatographic assay chamber being in fluid communication with the internal chamber downstream of the clot catcher;
detecting, by the chromatographic assay assembly, a presence of free hemoglobin in the fluid sample; and
transferring the fluid sample from the internal chamber to the liquid sample analyzer with the sample probe.

Illustrative embodiment 14. The illustrative method of embodiment 13, wherein the detecting step comprises:
applying the fluid sample to a sample application pad of the chromatographic assay assembly and allowing plasma and the free hemoglobin present in the fluid sample to flow through the sample application pad to a chromatographic detection pad while retaining red blood cells present the fluid sample in the sample application pad;
flowing, by capillary action, the plasma and the free hemoglobin from a sample application site of the chromatographic detection pad to a detection site of the chromatographic detection pad; and
visually comparing a color change at the detection site to a reference device containing a plurality of reference colors, wherein each reference color corresponds to a different level of hemolysis.

Illustrative embodiment 15. The illustrative method of any one of the preceding embodiments, wherein the sample application pad comprises a first layer and a second layer, and wherein the applying step further comprises saturating the first layer with the fluid sample, and passing the fluid sample from the first layer to the second layer to saturate the second layer with the fluid sample.

Illustrative embodiment 16. The illustrative method combination of any one of the preceding illustrative embodiments, wherein the chromatographic assay chamber is defined by a portion of an external surface of the barrel and a cover having a fill pane aligned with the sample application pad and a read pane aligned with the detection site of the chromatographic detection pad, and wherein the applying step further comprises viewing the sample application pad through the fill pane of the cover to determine whether the sample application is saturated with the fluid sample.

Illustrative embodiment 17. The illustrative method of any one of the preceding illustrative embodiments, wherein the visually comparing step comprises viewing the detection site through the read pane of the cover.

Illustrative embodiment 18. The illustrative method of any one of the preceding illustrative embodiments, wherein the step of transferring at least a portion the fluid sample from the fluid sample collection apparatus further comprises engaging the first end of the barrel with a portion of the fluid sample collection apparatus, and wherein the step of transferring the fluid sample from the internal chamber to the liquid sample analyzer further comprises engaging the second end of the barrel with the liquid sample analyzer so a combination of the fluid sample collection apparatus and the barrel is attached to the liquid sample analyzer with no additional support.

Illustrative embodiment 19. The illustrative method of any one of the preceding illustrative embodiments, wherein prior to transferring the fluid sample from the internal chamber to the liquid sample analyzer, determining that the detection of hemolysis of the visually comparing step is below a predetermined threshold of hemolysis.

Illustrative embodiment 20. The illustrative method of any one of the preceding illustrative embodiments, wherein the step of engaging the first end further comprises threading the first end of the barrel with the portion of the fluid sample collection apparatus so as to interlockingly engage the barrel with the fluid sample collection apparatus.

Illustrative embodiment 21. The illustrative method of any one of the preceding illustrative embodiments, wherein the first end of the barrel has a barrel connection portion that is a male luer connector, wherein the portion of the fluid sample collection apparatus has a female luer connector, and wherein the step of engaging the first end further comprises engaging the male luer connector of the barrel with the female luer connector of the fluid sample collection apparatus.

Illustrative embodiment 22. The illustrative method of any one of the preceding illustrative embodiments, wherein the second end of the barrel has a tubular portion that is a male luer connector, and wherein the step of engaging the second end comprises engaging the male luer connector of the barrel within a sample input port of the liquid sample analyzer.

Illustrative embodiment 23. The illustrative method of any one of the preceding illustrative embodiments, wherein the steps of engaging the first end and engaging the second end further comprises axially aligning the fluid sample collection apparatus and the barrel with the sample probe.

Illustrative embodiment 24. The illustrative method of any one of the preceding illustrative embodiments, wherein the apparatus further comprises a gas-permeable, liquid-impermeable membrane secured to the barrel adjacent the second end of the barrel, and wherein the step of transferring the fluid sample from the internal chamber of the barrel further comprises passing the sample probe through the gas-permeable, liquid-impermeable membrane to the internal chamber of the of the barrel.

Illustrative embodiment 25. The illustrative method of any one of the preceding illustrative embodiments, further comprising:
causing the fluid sample to contact a filter member disposed within the internal chamber, the filter member defining an inlet side and an outlet side of the internal chamber, the filter member having a gas-permeable, liquid-impermeable membrane;
separating at least a portion of the gas portion of the liquid sample from the liquid portion of the fluid sample by causing the fluid sample to contact the gas-permeable, liquid-impermeable membrane so that at least a portion of the gas portion of the fluid sample passes across the filter member from the inlet side to the outlet side of the internal chamber and so the liquid portion of the fluid sample is prevented from passing from the inlet side to the outlet side; and
collecting at least a portion of the liquid portion of the fluid sample from the inlet side of the internal chamber.

Illustrative embodiment 26. The illustrative method of any one of the preceding illustrative embodiments, wherein the step of collecting the fluid sample from the internal chamber of the barrel further comprises passing the sample probe through the gas-permeable, liquid-impermeable membrane to the inlet side of the internal chamber of the barrel.

What is claimed:

1. An apparatus for transferring a fluid sample having a liquid portion and a gas portion from a fluid sample collection apparatus to a liquid sample analyzer, comprising:
a barrel having a first end, a second end opposite the first end, a sidewall extending between the first end and the second end, an inner surface defining an internal chamber, and an external surface defining at least a portion of a chromatographic assay chamber in fluid communication with the internal chamber via a passage through the external surface of the barrel, the first end having an inlet opening having a clot catcher extending across the inlet opening upstream of the passage and the second end having an outlet opening; and
a chromatographic assay assembly housed in the chromatographic assay chamber configured to detect presence of free hemoglobin in the fluid sample, the chromatographic assay assembly comprising:
a sample application pad configured to receive the fluid sample from the internal chamber, wherein the sample application pad is formed of a first layer of a prefiltration material and a second layer of an asymmetric polysulfone material, wherein the sample application pad is porous to plasma and the free hemoglobin and not porous to red blood cells; and
a chromatographic detection pad in fluidic contact with the sample application pad, the chromatographic detection pad being configured to detect the presence of the free hemoglobin.

2. The apparatus of claim 1, wherein the chromatographic assay chamber is defined by a portion of the outer surface of the barrel and a cover, the cover having a transparent fill pane aligned with the sample application pad for viewing flow of the fluid sample through the sample application pad and a transparent read pane aligned with a detection site of the chromatographic detection pad for viewing the detection site.

3. The apparatus of claim 1, wherein the first end of the barrel has a barrel connection portion engageable with a portion of the fluid sample collection apparatus, and wherein the second end of the barrel has a tubular portion configured to engage with the liquid sample analyzer so a combination of the fluid sample collection apparatus and the barrel is attachable to the liquid sample analyzer with no additional support.

4. The apparatus of claim 3, wherein the barrel connection portion comprises at least one thread lug extending radially outwardly from the sidewall of the barrel so as to be interlockingly engageable with a portion of the fluid sample collection apparatus.

5. The apparatus of claim 3, wherein the barrel connection portion comprises a pair of thread lugs extending radially outwardly from the sidewall of the barrel so as to be interlockingly engageable with the fluid sample collection apparatus.

6. The apparatus of claim 3, wherein the barrel connection portion is a female luer connector.

7. The apparatus of claim 3, wherein the tubular portion of the barrel is a male luer connector configured to engage with the liquid sample analyzer.

8. The apparatus of claim 1, further comprising a gas-permeable, liquid-impermeable membrane secured to the barrel adjacent the second end of the barrel, the gas-permeable, liquid-impermeable membrane being pierceable so a sample probe is configured to be passed through the gas-permeable, liquid-impermeable membrane to the internal chamber.

9. The apparatus of claim 8, wherein the gas-permeable, liquid-impermeable membrane is formed from a material comprising at least one of polytetrafluorethylene, polypropylene, and polyethylene.

10. The apparatus of claim 1, further comprising:
a filter member disposed within the internal chamber so the filter member defines an inlet side and an outlet side of the internal chamber and so the filter member is positionable between the first end and the second end of the barrel, the filter member having at least one gas-permeable, liquid-impermeable membrane configured to permit at least a portion of the gas portion of the fluid sample to pass across the filter member from the inlet side to the outlet side of the internal chamber and to provide a fluid-tight seal across the filter member to prevent the liquid portion of the fluid sample from passing from the inlet side to the outlet side as the fluid sample is passed into the internal chamber via the inlet opening to separate at least a portion of the gas portion from the liquid portion of the fluid sample,
wherein the filter member is pierceable so a probe is configured to be passed through the filter member from the outlet side to the inlet side to withdraw the liquid portion of the fluid sample from the inlet side of the internal chamber.

11. The apparatus of claim 10, wherein the filter member is slidably disposed in the internal chamber of the barrel.

12. A kit, comprising:
the apparatus of claim 1; and
a reference device containing a plurality of reference colors, wherein each reference color corresponds to a different level of hemolysis.

13. A method of transferring a fluid sample having a liquid portion and a gas portion from a fluid sample collection apparatus to a liquid sample analyzer having a sample probe, the method comprising:
obtaining an apparatus having a barrel with a first end, a second end, a sidewall extending between the first end and the second end, and an inner surface defining an internal chamber, the first end having an inlet opening with a clot catcher extending across the inlet opening and the second end having an outlet opening;
transferring at least a portion the fluid sample from the fluid sample collection apparatus to the internal chamber of the barrel via the inlet opening so the fluid sample passes through the clot catcher at the inlet opening to capture solids in the fluid sample;
passing a portion of the fluid sample from the internal chamber of the barrel to a chromatographic assay chamber and a chromatographic assay assembly housed in the chromatographic assay chamber, the chromatographic assay chamber being in fluid communication with the internal chamber downstream of the clot catcher;
detecting, by the chromatographic assay assembly, a presence of free hemoglobin in the fluid sample; and
transferring the fluid sample from the internal chamber to the liquid sample analyzer with the sample probe.

14. The method of claim 13, wherein the detecting step comprises:
applying the fluid sample to a sample application pad of the chromatographic assay assembly and allowing plasma and the free hemoglobin present in the fluid sample to flow through the sample application pad to a chromatographic detection pad while retaining red blood cells present the fluid sample in the sample application pad;
flowing, by capillary action, the plasma and the free hemoglobin from a sample application site of the chromatographic detection pad to a detection site of the chromatographic detection pad; and
visually comparing a color change at the detection site to a reference device containing a plurality of reference colors, wherein each reference color corresponds to a different level of hemolysis.

15. The method of claim 14, wherein the sample application pad comprises a first layer and a second layer, and wherein the applying step further comprises saturating the first layer with the fluid sample, and passing the fluid sample from the first layer to the second layer to saturate the second layer with the fluid sample.

16. The method of claim 14, wherein the chromatographic assay chamber is defined by a portion of an external surface of the barrel and a cover having a fill pane aligned with the sample application pad and a read pane aligned with the detection site of the chromatographic detection pad, and wherein the applying step further comprises viewing the sample application pad through the fill pane of the cover to determine whether the sample application is saturated with the fluid sample.

17. The method of claim 16, wherein the visually comparing step comprises viewing the detection site through the read pane of the cover.

18. The method of claim 13, wherein the step of transferring at least a portion the fluid sample from the fluid sample collection apparatus further comprises engaging the first end of the barrel with a portion of the fluid sample collection apparatus, and wherein the step of transferring the fluid sample from the internal chamber to the liquid sample analyzer further comprises engaging the second end of the barrel with the liquid sample analyzer so a combination of the fluid sample collection apparatus and the barrel is attached to the liquid sample analyzer with no additional support.

19. The method of claim 18, wherein prior to transferring the fluid sample from the internal chamber to the liquid sample analyzer, determining that the detection of hemolysis of the visually comparing step is below a predetermined threshold of hemolysis.

20. The method of claim 18, wherein the step of engaging the first end further comprises threading the first end of the barrel with the portion of the fluid sample collection apparatus so as to interlockingly engage the barrel with the fluid sample collection apparatus.

21. The method of claim 20, wherein the first end of the barrel has a barrel connection portion that is a male luer connector, wherein the portion of the fluid sample collection apparatus has a female luer connector, and wherein the step of engaging the first end further comprises engaging the male luer connector of the barrel with the female luer connector of the fluid sample collection apparatus.

22. The method of claim 20, wherein the second end of the barrel has a tubular portion that is a male luer connector, and wherein the step of engaging the second end comprises engaging the male luer connector of the barrel within a sample input port of the liquid sample analyzer.

23. The method of claim 18, wherein the steps of engaging the first end and engaging the second end further comprises axially aligning the fluid sample collection apparatus and the barrel with the sample probe.

24. The method of claim 17, wherein the apparatus further comprises a gas-permeable, liquid-impermeable membrane secured to the barrel adjacent the second end of the barrel, and wherein the step of transferring the fluid sample from the internal chamber of the barrel further comprises passing the sample probe through the gas-permeable, liquid-impermeable membrane to the internal chamber of the of the barrel.

25. The method of claim 18, further comprising:
causing the fluid sample to contact a filter member disposed within the internal chamber, the filter member defining an inlet side and an outlet side of the internal chamber, the filter member having a gas-permeable, liquid-impermeable membrane;
separating at least a portion of the gas portion of the liquid sample from the liquid portion of the fluid sample by causing the fluid sample to contact the gas-permeable, liquid-impermeable membrane so that at least a portion of the gas portion of the fluid sample passes across the filter member from the inlet side to the outlet side of the internal chamber and so the liquid portion of the fluid sample is prevented from passing from the inlet side to the outlet side; and
collecting at least a portion of the liquid portion of the fluid sample from the inlet side of the internal chamber.

26. The method of claim 25, wherein the step of collecting the fluid sample from the internal chamber of the barrel further comprises passing the sample probe through the gas-permeable, liquid-impermeable membrane to the inlet side of the internal chamber of the barrel.
